(19) United States
(12) Patent Application Publication
(10) Pub. No.: US 2026/0021322 A1
(43) Pub. Date: Jan. 22, 2026

(54) MODULAR BOTTLE CAP WITH INTEGRATED PHYSIOTHERAPY COMPONENTS

(71) Applicant: SHENZHEN NUON MEDICAL EQUIPMENT CO., LTD., Shenzhen (CN)

(72) Inventors: Alain Dijkstra, Amstelveen (NL); LI XIANG, Shenzhen (CN); Tang Yuanzhou, Shenzhen (CN); Chen Kunjie, Shenzhen (CN); Meng Xiaoting, Shenzhen (CN)

(73) Assignee: SHENZHEN NUON MEDICAL EQUIPMENT CO., LTD., Shenzhen (CN)

(21) Appl. No.: 19/243,772

(22) Filed: Jun. 20, 2025

(30) Foreign Application Priority Data
Jul. 18, 2024 (CN) 2024109627430
Jul. 18, 2024 (CN) 2024217188784

(51) Int. Cl. A61N 5/06; B65D 51/24

(52) U.S. Cl. CPC A61N 5/0616; B65D 51/24; A61N 2005/0626; A61N 2005/0632; A61N 2005/0644

(57) ABSTRACT

Embodiments of the present invention provide a modular bottle cap with an integrated physiotherapy component. The modular bottle cap comprising a physiotherapy device host and a storage shell configured to attach to a container body. The physiotherapy device host includes a housing with one or more stimulation elements—such as phototherapy, micro-current, vibration, or ultrasonic components—controlled by an internal circuit assembly. The device further integrates a detection probe to monitor skin parameters like moisture, oil, and temperature, allowing real-time, adaptive therapy through a control unit. A user interface, optionally featuring a touchscreen, enables the selection of treatment modes, and intensity adjustments, and displays live feedback.

### TECHNICAL FIELD

The present invention relates to the technical field of skincare devices and personal care product containers. More particularly, it pertains to a multifunctional modular bottle cap structure integrated with a physiotherapy device host. Specifically, the invention provides a bottle cap configured to house and store a physiotherapy module capable of performing one or more therapy, sensing skin parameters, and display functions. The bottle cap is designed for convenient storage, modular assembly, and compatibility with a variety of cosmetic and skincare containers, thereby enhancing portability and user convenience in daily skincare routines.

### BACKGROUND

Phototherapy is a skincare technique that utilizes light of specific wavelengths to act on the skin's surface. This technology operates on the principle that biological tissues, such as cells and mitochondria, can absorb light energy within certain wavelength ranges. Once absorbed, this light energy is converted into thermal or chemical energy, thereby initiating a cascade of biochemical reactions. These reactions promote blood circulation, accelerate cellular metabolism, enhance skin vitality, and increase the absorption efficiency of topical skincare products. As a result, phototherapy has gained widespread application in the fields of skincare, cosmetic treatment, and health maintenance.

Conventional phototherapy devices are often compact and portable, making them suitable for everyday skincare routines. Typically, users apply serums, creams, or lotions to the skin and then irradiate the treated area with light of a specific wavelength using a phototherapy device. This process facilitates deeper penetration of active ingredients, enhances the efficacy of skincare products, and improves the overall user experience.

However, most existing phototherapy devices are designed as standalone units, physically separate from skincare product containers. During use, the user must locate and operate the device independently, which adds steps to the skincare routine and reduces efficiency. Moreover, the small size of these devices makes them susceptible to being misplaced or lost, diminishing convenience and practicality in daily use.

This issue is further exacerbated in scenarios such as travel, business trips, or outdoor use, where users prefer lightweight and multifunctional skincare products. The need to carry a separate phototherapy device increases luggage load and contradicts the growing demand for integrated, portable skincare solutions.

Accordingly, there is a pressing need for an improved skin phototherapy device that is structurally integrated or conveniently attachable to a cosmetic container or skincare bottle. Such a solution would streamline the skincare process, improve portability, and significantly enhance user convenience and experience.

### OBJECTS OF THE INVENTION

Some of the objects of the invention are as follows:

An object of the present invention is to provide a multifunctional modular bottle cap integrated with a physiotherapy device host, thereby enhancing portability, ease of storage, and convenience in performing skincare treatments.

Another object of the invention is to provide a bottle cap structure comprising a storage shell with a housing configured to accommodate a modular physiotherapy device host, allowing for simple assembly, disassembly, and secure storage of the device.

A further object of the invention is to provide a physiotherapy device host integrated with one or more stimulation elements to perform one or more therapies that include but are not limited to phototherapy, electrical pulse massage, vibration massage therapy.

Another object of the invention is to provide a physiotherapy device host that can be reliably positioned and secured within the storage shell using a card slot and magnetic snap-fit mechanism, ensuring stable fixation and preventing accidental detachment during use or transport.

Yet another object of the invention is to provide a modular bottle cap structure equipped with an adapter featuring internal threads, allowing the cap to be securely mounted onto various types and sizes of cosmetic or skincare containers through bonding, clamping, or threaded connection methods.

Still, another object of the invention is to provide a multifunctional physiotherapy bottle cap that incorporates intelligent skin parameter sensing capabilities, enabling real-time regulation of the therapy functions to improve treatment precision and skincare efficacy.

### SUMMARY OF THE INVENTION

According to a first aspect of the present invention, a system for providing one or more therapy to a user is provided. The system comprising: a physiotherapy device host, comprising: a housing; one or more stimulation elements disposed within the housing configured to provide one or more therapy to the user; a circuit assembly electrically connected to the one or more stimulation elements for regulating operations; a storage shell having a first end for attachment to a container body and a second end having a storage slot configured to receive the physiotherapy device host in a detachable manner; wherein the physiotherapy device host is configured to modularly fit in the storage shell.

In one embodiment of the invention, the one or more stimulation elements are selected from the group consisting of a phototherapy component, micro-current component, heating element, cooling element, Peltier element, vibrational element for massage, and ultrasonic wave component.

In one embodiment of the invention, the storage slot includes a securing mechanism selected from the group consisting of a snap-fit mechanism, magnetic attachment, friction fit, or threaded engagement.

In one embodiment of the invention, the physiotherapy device host further comprises a rechargeable power source electrically connected to the circuit assembly.

In one embodiment of the invention, the circuit assembly includes a wireless communication module for connectivity with external devices.

In one embodiment of the invention, the storage shell is magnetically attracted or snap-fitted to the physiotherapy device host for secure attachment.

In one embodiment of the invention, the storage shell includes a first notch and a second notch disposed opposite to each other to accommodate components of the physiotherapy device host.

In one embodiment of the invention, the storage shell includes a split upper shell and a transfer piece, the upper shell is provided with the storage slot, and the transfer piece includes a mounting position to secure the device.

According to a second aspect of the present invention, a system for providing one or more therapy to a user is provided. The system comprising: a physiotherapy device host having a front shell section and a rear shell section, the front shell section is wider than the rear shell section, the front shell section is provided with a light-transmitting portion, the physiotherapy device comprising: a housing; one or more stimulation elements disposed within the housing to provide one or more therapy to a user; at least one detection probe to detect one or more skin parameters; a circuit assembly electrically connected to the one or more stimulation element for controlling therapy output; a storage shell having a first end for attachment to a container body and a second end having a storage slot configured to detachably receive the physiotherapy device host.

In one embodiment of the invention, the at least one detection probe is selected from the group consisting of a temperature sensor, moisture sensor, impedance sensor, optical sensor, or a combination thereof.

In one embodiment of the invention, the detection probe is configured to detect at least one of the water level, oil level, temperature, or hydration levels of the user's skin.

In one embodiment of the invention, the light-transmitting portion comprises a transparent or semi-transparent material configured to diffuse light evenly.

In one embodiment of the invention, the storage shell includes an adapter configured to accommodate container bodies of different sizes.

In one embodiment of the invention, the storage shell further comprises a secondary compartment for storing additional accessories, such as replacement therapy heads or power cables.

In one embodiment of the invention, the light-transmitting portion houses metal electrodes that are electrically connected to the main control board for delivering micro-current therapy.

According to a third aspect of the present invention, a system for providing one or more therapy to a user is provided. The system comprising: a physiotherapy device host, comprising: a detection sensor configured to detect one or more skin parameters of a user; one or more stimulation elements configured to provide one or more therapies to the user; a control unit configured to: regulate the operation of the one or more stimulation element based on predefined treatment parameters; adjust the output of the one or more stimulation elements dynamically in response to real-time skin parameters; a user interface having at least one input control to allow a user to select treatment modes, adjust intensity levels, and activate or deactivate the one or more stimulation element.

In one embodiment of the invention, the detection sensor is configured to analyze skin hydration levels and adjust therapy parameters accordingly.

In one embodiment of the invention, the control unit is programmed with multiple treatment modes, including pre-set therapy cycles based on skin conditions.

In one embodiment of the invention, the user interface includes a touchscreen display for real-time monitoring of therapy settings and skin parameter data.

In one embodiment of the invention, the control unit is configured to store user treatment history and provide customized therapy recommendations.

In one embodiment of the invention, the system further comprises a mobile application that allows a user to control the physiotherapy device remotely via Bluetooth or Wi-Fi connectivity.

In one embodiment of the invention, the user interface includes a touchscreen display configured to provide visual feedback on treatment settings and progress.

In one embodiment of the invention, the physiotherapy device host features an automatic shut-off function after a predefined therapy duration to prevent overuse.

In the context of this specification, terms like “light”, “radiation”, “irradiation”, “emission” and “illumination”, etc. refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for treatment of conditions that occur at the skin surface, such as psoriasis or infection.

In the context of the specification, the term “light source” or “phototherapy source” etc. refers to a source emitting coherent laser light, or light-emitting diodes (“LEDs”). The term “light therapy” refers to light generated from any of the sources, such as laser, LED sources, or Super luminous diodes (“SLD”).

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first’, “second” and “third” are only used for descriptive purposes and do not implicate the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light and Magnesium-doped potassium fluorosilicate in combination with a blue LED to generate red light.

In the context of the specification, the term “physiotherapy device host” broadly includes any handheld or modular unit capable of delivering one or more therapeutic functions.

In the context of the specification, the term “storage shell” may include any container, compartment, or docking structure designed to hold, store, or interface with the physiotherapy device.

In the context of the specification, the term “Stimulation elements” may include, but are not limited to, components that deliver light, heat, vibration, electrical pulses, or other forms of therapeutic output.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 illustrates a perspective view of a modular bottle cap, in accordance with an embodiment of the present invention.

FIG. 2 depicts an exploded top view of a storage shell of the modular bottle cap, in accordance with an embodiment of the present invention.

FIG. 3 illustrates an exploded bottom view of the storage shell, in accordance with an embodiment of the present invention.

FIG. 4 represents a cross-sectional view of the storage shell, in accordance with an embodiment of the present invention.

FIG. 5 illustrates a top-perspective view of a physiotherapy device host integrated with the bottle cap, in accordance with an embodiment of the present invention.

FIG. 6 illustrates a bottom perspective view of the physiotherapy device host, in accordance with an embodiment of the present invention.

FIG. 7 depicts a cross-sectional view of the physiotherapy device host, in accordance with an embodiment of the present invention.

FIG. 8 is an exploded view of the physiotherapy device host, in accordance with an embodiment of the present invention.

FIG. 9 illustrates an exploded view of a front housing of the physiotherapy device host, in accordance with an embodiment of the present invention.

FIG. 10 illustrates a perspective view of the structure of the modular bottle cap and a corresponding cosmetic container, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention disclose a modular bottle cap configured to adapt to a bottle head and to provide one or more treatment to a user. The modular bottle cap comprises a physiotherapy device host and a corresponding storage shell, enabling compact integration, case of use, and multifunctional treatment options. The device is particularly suitable for skincare and cosmetic applications where targeted therapy and portability are desired.

The physiotherapy device host includes a compact housing that is ergonomically designed and comprises a front shell section and a rear shell section. The front shell section is relatively wider and includes a light-transmitting portion for delivering optical therapy. The housing is configured to contain and support a variety of functional components, including stimulation elements, detection probes, a circuit assembly, a rechargeable power source, and a user interface. The physiotherapy device host comprises one or more stimulation elements disposed within the housing. These elements are configured to deliver different types of therapies, including but not limited to Phototherapy components, Micro-current components for electrical stimulation, Heating or cooling elements, Vibrational components for massage therapy, and Ultrasonic wave components for deep tissue or pore-level treatment. These components are operatively connected to the internal circuit assembly, which regulates their operation based on user input or sensor feedback.

The physiotherapy device host further comprises at least one detection probe integrated into the device for real-time monitoring of skin parameters. The probe may include sensors such as moisture sensors, temperature sensors, impedance sensors, and/or optical sensors. These are used to detect values such as hydration levels, oil content, or skin temperature, enabling personalized therapy adjustments.

The detection end of the probe is exposed on the outer surface of the housing and is placed at the end of the rear shell section. This arrangement helps in space optimization and allows targeted contact with the skin.

The circuit assembly includes a flexible or rigid printed circuit board equipped with a main control unit and power management circuitry. It may further incorporate a wireless communication module for connectivity with external devices such as smartphones or tablets via Bluetooth or Wi-Fi.

The control unit is programmed to: operate in multiple pre-defined or customizable therapy modes; dynamically adjust stimulation parameters in response to real-time sensor inputs; store user data and treatment history for personalized recommendations; control charging and power delivery from the rechargeable battery. An automatic shut-off function is included to prevent overuse by terminating therapy after a predefined duration.

The housing further comprises a user interface that includes a touchscreen display for displaying real-time data and controls, a key or button switch for basic operational commands, and integrated charging components within the key switch to allow direct contact charging when docked in the storage shell.

The storage shell of the modular bottle cap acts as a docking unit for the physiotherapy device host. One end of the storage shell is configured to attach to a cosmetic container body, allowing integrated packaging of the device with skincare products.

The storage shell comprises a first end that attaches to a container and a second end with a storage slot to securely hold the device. The slot may incorporate a snap-fit, magnetic, friction, or threaded securing mechanism to retain the device host.

The shell includes first and second notches located on opposite sides to accommodate protruding components such as the detection probe and key switch, ensuring a flush and stable fit.

Several additional functionalities may also be added to the wearable phototherapy hair growth device. For example, the light-transmitting portion may house metal electrodes for delivering micro-current stimulation. The device is modular, allowing different components or therapy heads to be swapped. A mounting bracket within the device supports key components such as the circuit board, display, and therapy elements, ensuring a compact and organized internal layout. A secondary compartment within the storage shell may be provided for storing accessories such as replacement therapy heads or power cables. The system may be operated or configured via a dedicated mobile application that enables remote control, data viewing, and therapy customization.

Several embodiments of the present invention will now be described in detail with references to FIGS.

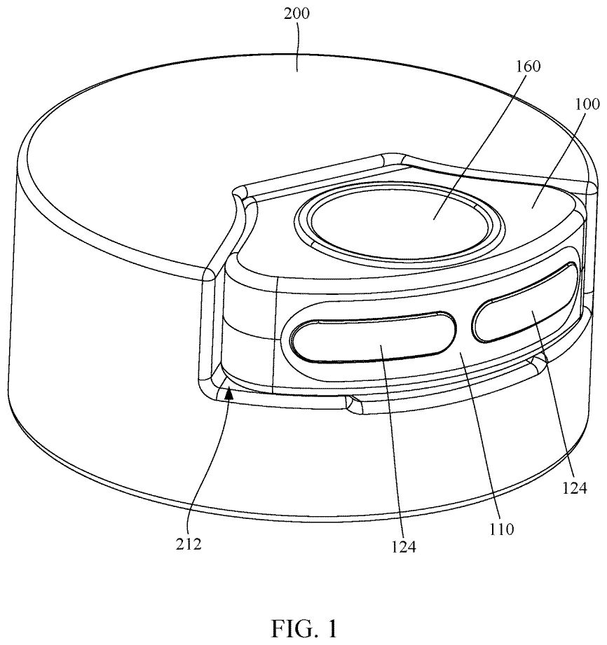

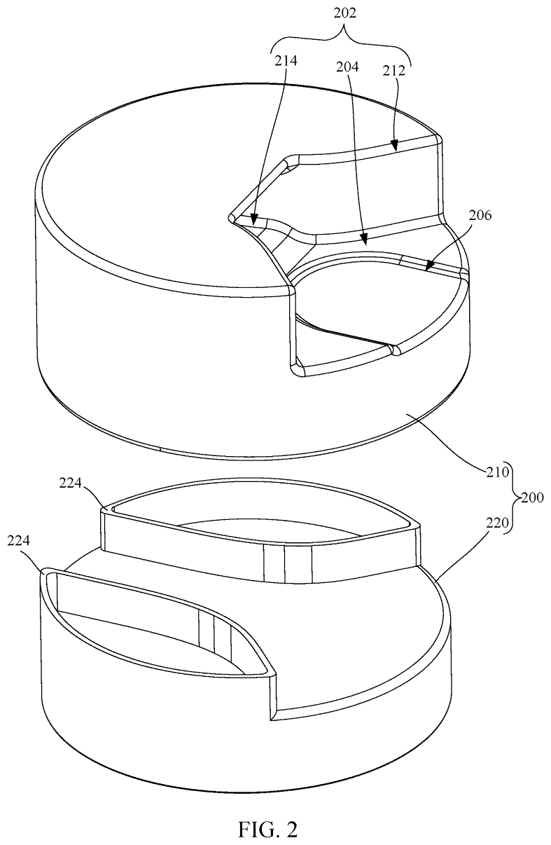

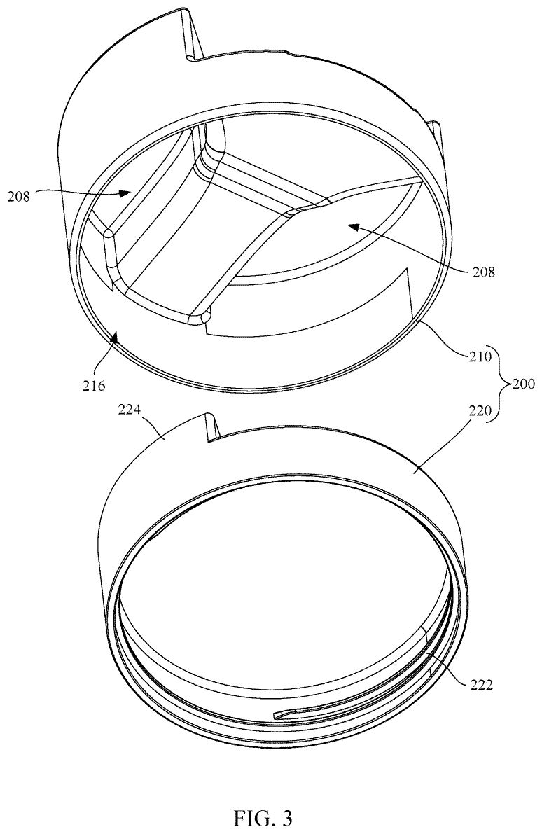

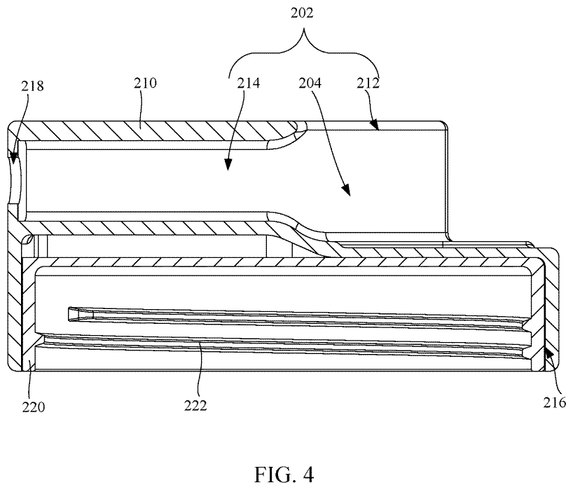

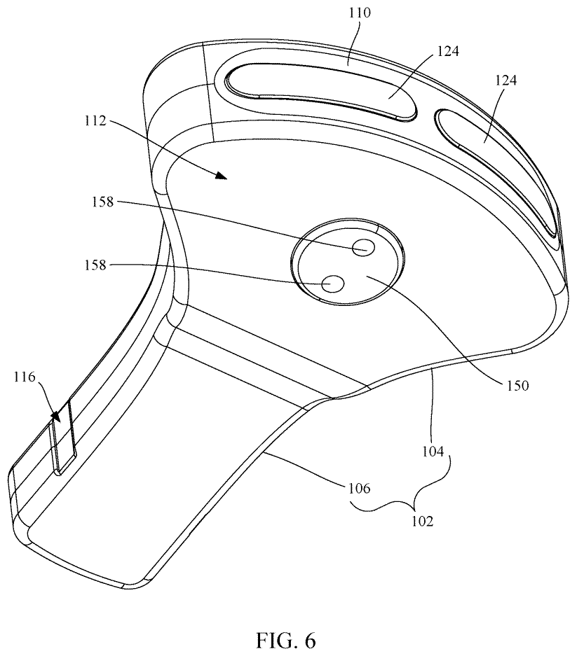

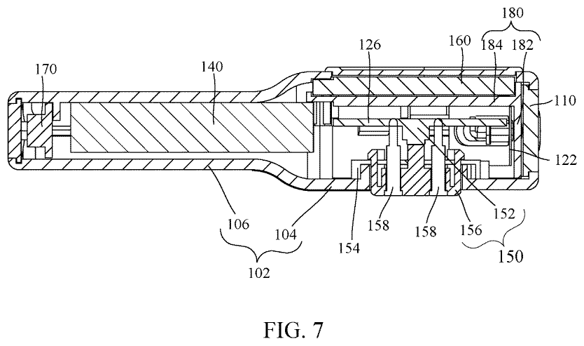

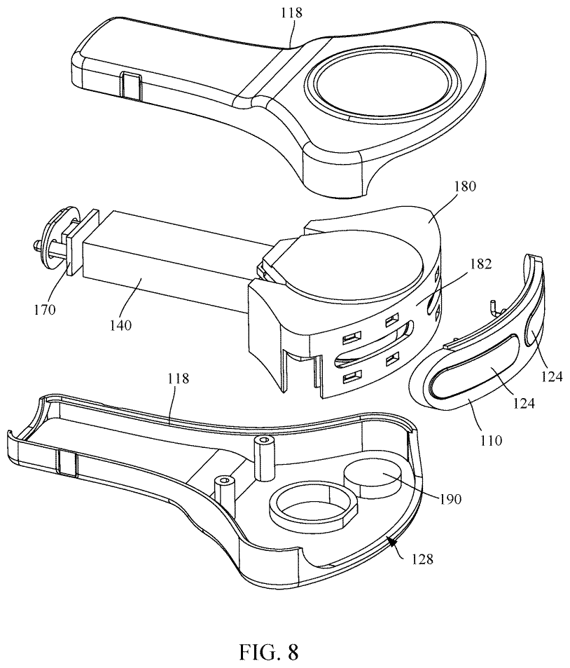

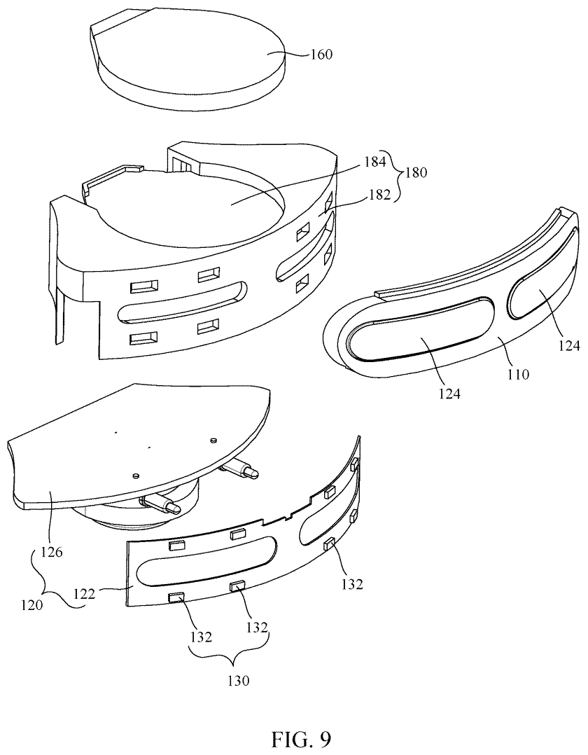

Referring to FIGS. 1 to 9, a modular bottle cap is disclosed, comprising a physiotherapy device host 100 and a storage shell 200. The physiotherapy device host 100 includes a housing 102 and at least one stimulation element 130. The physiotherapy device host 100 includes one or more stimulation elements 130. The housing 102 is provided with a light-transmitting portion 110, and the stimulation elements 130 are present in the housing 102 in alignment with the light-transmitting portion 110. When the stimulation element is a phototherapy component, this configuration enables the phototherapy component to emit light of a predetermined wavelength outward through the light-transmitting portion 110 for topical application to the skin.

The storage shell 200 is configured with a mounting position for securing the device to the container mouth or cap of a cosmetic container and further includes a storage groove 202 designed to accommodate the physiotherapy device host 100. The physiotherapy device host 100 is operable between two states: a storage state, in which the device is inserted and retained within the storage groove 202, and a separated state, in which the device is detached from the storage shell 200 for independent use.

In an embodiment of the present invention the one or more stimulation elements 130 is selected from the group consisting of but is not limited to a laser, a heating element, a cooling element, a vibration element, electrodes, a micro-current element, an ultrasonic wave component or a combination thereof. The vibration element can be used as a massage head to massage the skin of a user, the micro-current element is used to impart, micro-current, the heating element, and the cooling element are used to provide heating and cooling effect to the skin of the user, and the ultrasonic wave component for deep tissue or pore-level treatment.

In the case of stimulation element 130 being a phototherapy component, the phototherapy component includes at least one light-emitting element 132 configured to emit light of a specific wavelength suitable for cosmetic treatment. During operation, light emitted from the light-emitting elements 132 passes through the light-transmitting portion 110 and irradiates the user's skin to achieve therapeutic or cosmetic effects.

The phototherapy component may include one or more types of light-emitting elements 132, which may emit red light, blue light, purple light, or other wavelength ranges as required for targeted skincare treatments. The physiotherapy device host 100 also comprises a control unit 120 disposed within the housing 102 and electrically connected to the phototherapy component for controlling its activation and operation.

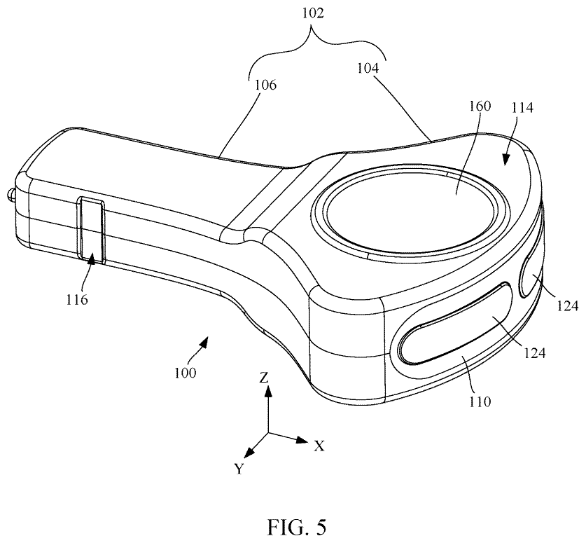

For orientation purposes, the length direction of the housing 102 or the physiotherapy device host 100 corresponds to the direction indicated by arrow X in FIG. 5, the width direction corresponds to the direction indicated by arrow Y, and the thickness direction corresponds to the direction indicated by arrow Z.

The storage shell 200 may be mounted onto the mouth or cap of the cosmetic container via the designated mounting position. Once mounted, the physiotherapy device host 100 can be securely inserted and retained within the storage groove 202. This structural configuration enables integrated storage and transport of the physiotherapy device host 100 along with the cosmetic container, thereby reducing the need for additional accessories and enhancing the portability and convenience of daily skincare routines. Upon application of skincare products from the container, the user can readily retrieve the physiotherapy device host 100 for immediate use, eliminating the inconvenience of locating a separate treatment device.

The storage groove 202 is configured to accommodate and securely store the physiotherapy device host 100, while the mounting position enables the storage shell 200 to be reliably affixed to a cosmetic container. Through this integrated structural arrangement, the skin phototherapy device is assembled in conjunction with the skincare product container, allowing the user to conveniently retrieve the physiotherapy device host 100 immediately after applying skincare products. This facilitates subsequent skin irradiation, thereby enhancing the absorption of the applied skincare formulation. Such integration effectively eliminates the inconvenience associated with locating and handling a separate phototherapy device.

Furthermore, by enabling the skin phototherapy device to be carried together with the cosmetic container, the user is afforded the convenience of performing phototherapy treatments at any time following the application of skincare products. This not only promotes better absorption but also significantly enhances the overall user experience and treatment convenience.

The storage groove 202 is further provided with a first notch 212 located on the outer surface of the storage shell 200. The first notch 212 is spaced apart from the mounting position and is configured to receive and release the physiotherapy device host 100 in a detachable manner. The spatial separation between the first notch 212 and the mounting position ensures that when the storage shell 200 is installed onto the container body, the assembly and disassembly of the physiotherapy device host 100 are not obstructed. This design improves accessibility without compromising mounting integrity.

To enhance stability during storage or transportation, the physiotherapy device host 100 may be retained within the storage groove 202 using a snap-fit mechanism or magnetic attachment. This ensures secure fixation and prevents unintended dislodgment or loss of the device during routine handling or movement.

The storage shell 200 is formed with an open end, and an internal thread 222 is provided along its inner wall to define the mounting position. Through this configuration, the storage shell 200 can be directly screwed onto the mouth of the container body, functioning as a secure container lid. Alternatively, the storage shell 200 may be sleeved over an existing lid of the cosmetic container. This dual-purpose design contributes to reducing the overall size and height of the assembled unit, thereby improving portability and structural compactness.

The storage shell 200 comprises an upper shell 210 and an adapter 220. The upper shell 210 is configured with the storage groove 202 to house the physiotherapy device host 100, while the adapter 220 is assembled onto the upper shell 210 and defines the mounting position. The upper shell 210 and adapter 220 are independently molded and subsequently joined, allowing the phototherapy cap to be adapted to cosmetic containers of varying specifications by simply modifying the adapter 220, thereby reducing tooling complexity and production cost. Alternatively, the storage groove 202 and the mounting position may also be integrally formed during the molding of the storage shell 200. The adapter 220 includes an open end, with an internal thread 222 formed along its inner wall to engage with the container body.

To ensure secure assembly, the upper shell 210 may be formed with a connection groove 216 on the side opposite the storage groove 202. The adapter 220 is inserted into the connection groove 216 and concentrically aligned with the upper shell 210. This configuration increases the contact area between the adapter 220 and the upper shell 210, thereby enhancing the stability and reliability of their connection. The adapter 220 may be affixed to the upper shell 210 through methods such as bonding, clamping, screwing, or the use of adhesives applied within the connection groove 216.

The housing 102 of the physiotherapy device host 100 comprises a front shell section 104 and a rear shell section 106, wherein the width of the front shell section 104 is greater than that of the rear shell section 106. The front shell section 104 is provided with a light-transmitting portion 110 and internally houses one or more stimulation elements 130 and the control unit 120. This configuration allows the functional components to be centrally positioned within the front shell section 104, enabling the rear shell section 106 to adopt a reduced size. The smaller dimensions of the rear shell section 106 facilitate easier accommodation and secure retention within the storage groove 202 of the storage shell 200, thereby reducing spatial constraints imposed by the larger front shell section 104.

The storage shell 200 is formed with an open end, and an internal thread 222 is provided on its inner wall to define a mounting position. This configuration allows the storage shell 200 to be directly threaded onto the mouth of a container body, enabling it to function as a container lid. Compared to designs where the storage shell is mounted on a separate lid, this integrated configuration effectively reduces the overall size of the skin phototherapy device when assembled with the container. In one embodiment, the storage shell 200 may also be sleeved over an existing container lid.

In an embodiment, the storage shell 200 comprises an upper shell 210 and an adapter 220. The upper shell 210 is formed with a storage groove 202 for housing the physiotherapy device host 100, while the adapter 220 is assembled onto the upper shell 210 and includes the mounting position. During manufacturing, the upper shell 210 and adapter 220 are independently molded and subsequently assembled, thereby allowing the phototherapy device to be easily adapted to containers of various sizes by simply modifying the adapter 220, without altering the mold of the upper shell 210. This modular design reduces manufacturing complexity and production costs. In alternative embodiments, the storage groove 202 and the mounting position may be integrally formed within the storage shell 200.

In an embodiment, the adapter 220 has an open end and includes an internal thread 222 defining the mounting position. The upper shell 210 may be formed with a connection groove 216 on the side opposite the storage groove 202, into which the adapter 220 is assembled. The adapter 220 may be positioned either internally or externally relative to the upper shell 210, increasing the contact area between components and enhancing assembly stability. The adapter 220 may be secured to the upper shell 210 using bonding, clamping, screwing, or adhesive fixation at the connection groove 216.

In certain embodiments, the housing 102 of the physiotherapy device host 100 comprises a front shell section 104 and a rear shell section 106 connected thereto. The front shell section 104 has a greater width than the rear shell section 106 and includes the light-transmitting portion 110. The one or more stimulation elements 130 and the control unit 120 are both housed within the front shell section 104, allowing the rear shell section 106 to be downsized. This compact rear shell design enables easier insertion into the storage groove 202 and facilitates reduction in the overall size of both the storage shell 200 and the physiotherapy device host 100 when stored.

The storage groove 202 includes a first slot section 204 and a second slot section 214 connected thereto. The first slot section 204 is provided with a first notch 212, and the second slot section 214 has a smaller width than the first slot section 204. The first slot section 204 is sized to receive the front shell section 104, while the second slot section 214 is configured to accommodate the rear shell section 106. This tiered structure ensures a snug fit, facilitating easy assembly and disassembly while minimizing clearance between corresponding parts, thereby enhancing overall structural compactness.

In an embodiment, the rear shell section 106 is provided with a slot 116, and a corresponding cam structure may be arranged within the first slot section 204. When the physiotherapy device host 100 is inserted into the storage groove 202, the cam engages with slot 116 to form a snap-fit connection. Alternatively, the rear shell section 106 may closely engage the second slot section 214, wherein its width or thickness slightly exceeds that of the second slot section 214, thereby generating frictional retention to secure the device in place.

The first notch 212 extends through both the circumferential wall and the outer surface of the storage shell 200, opposite the mounting position. This extended notch design facilitates easier insertion of the physiotherapy device host 100 into the storage groove 202. Additionally, by reducing the wall thickness between the second slot section 214 and the external surface of the storage shell 200, this configuration contributes to minimizing the overall height of the storage shell 200 and the combined assembly size.

The light-transmitting portion 110 is positioned on the end face of the front shell section 104, opposite the rear shell section 106. This arrangement enables the user to grip the rear shell section 106 and apply the physiotherapy device host 100 perpendicularly to the skin surface, thereby improving handling comfort and operational convenience. Furthermore, this orientation optimizes the use of space on the front shell's end face, contributing to a more compact device design.

The width of the front shell section 104 tapers progressively toward the rear shell section 106, resulting in a smooth external contour that enhances ergonomic handling. This tapered form not only improves the user's grip but also optimizes internal space allocation while maintaining a streamlined external profile.

In an embodiment, a positioning groove 208 is formed on the bottom wall of the connection groove 216, offset from the storage groove 202. The adapter 220 is provided with a corresponding positioning protrusion 224, which extends into the positioning groove 208 to ensure accurate alignment during assembly. Specifically, the upper shell 210 may be formed from a plastic material via injection molding, enabling positioning grooves 208 to be formed on opposing sides of the first slot section 204 to enhance positioning accuracy.

The physiotherapy device host 100 further includes a battery 140, which is disposed within the rear shell section 106 and electrically connected to the control unit 120. The device may be powered by the battery 140 without requiring an external power supply, thereby enhancing portability and user convenience.

The housing 102 is provided with a first surface 112 and a second surface 114, spaced apart along the thickness direction of the housing 102. Both surfaces are formed with mounting holes. The physiotherapy device host 100 further comprises a key switch 150 disposed within one of the mounting holes. Two charging components 158 are embedded within the key switch 150 and are electrically connected to the control unit 120.

The control unit 120 includes a main control board 126, and the key switch 150 comprises a press switch 152 and a key cap 156. The press switch 152 is mounted on the main control board 126, while the key cap 156 is positioned within the mounting hole. The two charging components 158 are embedded within the key cap 156. When the charging components 158 are connected to a charger, the battery 140 can be recharged. The integration of the charging components 158 within the key switch 150 allows the key switch's rebound feature to maintain stable contact with the charger during charging, eliminating the need for a separate magnetic or adsorption mechanism. This configuration simplifies the overall structure of the physiotherapy device host 100 and contributes to a more compact design.

In an embodiment of the present invention, the charging component can be positioned on the storage shell 200 to facilitate charging of the rechargeable battery of the physiotherapy device host 100.

A display assembly 160 is disposed on the second surface 114 and is electrically connected to the control unit 120. The key switch 150 and the display assembly 160 are positioned on opposite sides of the housing 102 (i.e., the front shell section 104), thereby optimizing internal space utilization within the housing 102.

In an embodiment of the present invention, the display assembly 160 can be positioned on the storage shell 200. The display assembly 160 is communicatively coupled to electronics accessories and sensors present in the physiotherapy device host 100. By integrating display in the storage shell 200, it eliminates the need of any external device for displaying the analysis.

In an embodiment, the physiotherapy device host 100 comprises a user interface with key buttons to control and regulate the functioning of the one or more stimulation elements 130. The key buttons on the user interface can be manually operated by the user to control the operations of the one or more stimulation elements 130.

To further reduce the overall size of the physiotherapy device, the rear shell section 106 is configured with reduced dimensions. Accordingly, the portion of the storage groove 202 corresponding to the rear shell section 106 (i.e., the second slot section 214) is also formed with a reduced size. This configuration facilitates secure fixation of the rear shell section 106 while enabling simplification of the structural design of the portion of the storage groove 202 corresponding to the front shell section 104 (i.e., the first slot section 204).

For example, the first notch 212 may extend through both the peripheral surface and the surface of the storage shell 200 opposite the mounting position. This enlarged opening facilitates the convenient insertion of the physiotherapy device host 100 into the storage groove 202. The display assembly 160 includes a display screen that is electrically connected to the control unit 120 and covered by a transparent protective cover mounted to the housing 102. This cover ensures the visibility of the display while maintaining the structural integrity and durability of the device.

An avoidance slot 206 is formed along the wall of the storage groove 202 in alignment with the physiotherapy device host 100. The orientation of the physiotherapy device host 100 within the storage groove 202 is such that the key switch 150 or the display assembly 160 is received within the avoidance slot 206 when in the stored state. This design prevents interference between these components and the groove wall during insertion or removal, ensuring smooth operation.

Moreover, when the first notch 212 extends through both the circumferential surface and the rear-facing surface of the storage shell 200, the physiotherapy device host 100 may be stored with the key switch 150 oriented either upwards or downwards. This bidirectional insertion capability enhances case of use and improves user convenience.

In an embodiment, a sealing silicone element is disposed at a position corresponding to the key switch 150 within the housing 102. This configuration enhances the seal between the key switch 150 and the housing 102, thereby improving water resistance and reducing the risk of moisture ingress into the interior of the device.

The physiotherapy device host 100 further comprises a detection probe 170 configured to measure one or more skin parameters. The one or more skin parameters include the skin's moisture and oil levels. The detection end of the detection probe 170 is exposed externally from the housing 102 and is electrically connected to the control unit 120. The detection probe 170 includes at least two detection electrodes for applying a low-level current to the skin, enabling resistance-based measurements to determine hydration levels. This facilitates real-time skincare diagnostics, allowing users to make informed skincare decisions.

In one embodiment, the detection probe 170 is positioned at the distal end of the rear shell section 106, opposite the front shell section 104. This placement utilizes internal space efficiently without increasing the overall size of the device.

In further embodiments, the storage groove 202 comprises a first notch 212 and a second notch 218 disposed on opposite sides. The physiotherapy device host 100 is inserted through the first notch 212, while the detection probe 170 is exposed through the second notch 218. The second notch 218 is aligned with the detection probe 170, eliminating the need for a reserved gap between the probe and the groove wall, thereby reducing the overall dimensions of the storage shell 200. Additionally, the second notch 218 may be formed on the peripheral surface of the storage shell 200.

The peripheral surface of the storage shell 200 is cylindrical, with the first notch 212 formed on this surface. Correspondingly, the end portion of the housing 102 is also cylindrical. In the storage state, the cylindrical surface of the housing 102 aligns flush with the peripheral surface of the storage shell 200. Specifically, the end surface of the front shell section 104, facing away from the rear shell section 106, is cylindrical and abuts the storage shell 200 upon insertion, preventing protrusion and enhancing portability and visual appeal.

In one embodiment, the one or more stimulation elements 130 include a micro-current electrode and an electric pulse massage. The physiotherapy device host 100 includes two metal electrodes 124 embedded within the light-transmitting portion 110 and partially exposed. These electrodes are electrically connected to the control unit 120, which includes an electric pulse massage circuit. When in contact with the skin, the electrodes deliver pulsed electrical stimulation to provide a therapeutic massage effect.

The control unit 120 comprises a flexible circuit board 122, and the light therapy module includes a plurality of light-emitting elements 132 mounted thereon. The front shell section 104 is formed with an end wall that defines the light-transmitting portion 110. The flexible circuit board 122 is mounted within this end wall, conforming to its curvature to improve light projection and transmission efficiency.

An assembly hole 128 may be formed at the distal end of the front shell section 104, opposite the rear shell section 106. A light-transmitting plate is installed within the assembly hole to protect internal components and enhance waterproofing while simplifying the manufacturing process. In an alternative embodiment, through-holes corresponding to each light-emitting element may be formed, or the light-transmitting portion 110 may be integrally molded with the front shell section 104.

Both the first surface 112 and the second surface 114 of the front shell section 104 are convex in the thickness direction, enabling the rear shell section 106 to be received within a complementary storage groove 202 formed in the storage shell 200. This contributes to a snug fit and compact structural profile.

The physiotherapy device host 100 may also be equipped with a vibrator 190, mounted to the inner wall of the housing 102 and electrically connected to the control unit 120. The vibrator 190 provides vibration-based massage functionality, further enhancing the user experience.

A mounting bracket 180 is arranged within the front shell section 104 to support the display assembly 160, one or more stimulation elements 130, and the control unit 120. This centralized component arrangement results in a compact internal structure, reducing the overall volume of the device and improving case of storage and handling. The housing 102 may be composed of two interlocking half-shells 118 for convenient assembly.

The mounting bracket 180 includes a first support plate 184 and a second support plate 182. The physiotherapy device host 100 also comprises a key switch 150, display assembly 160, and battery 140. The control unit 120 includes a main control board 126 and the flexible circuit board 122. These components—including the key switch 150, display assembly 160, and the first support plate 184—are arranged along the thickness direction of the front shell section 104. The display assembly 160 is mounted on the first support plate 184, contributing to a compact and efficient internal layout.

In an embodiment of the present invention, the physiotherapy device host 100 is configured to connect with an external communication device over a communication interface. The control unit 120 includes a processor, a memory unit, and a communication interface. The communication with the external communication device is routed through the communication interface. The control unit is configured to connect with the external communication device and receives an input. The input may correspond to activating the light therapy module for phototherapy or controlling one or more parameters of operating the light therapy module, the micro-current electrode (metal electrodes 124), the vibrator 190, and the one one or more stimulation elements 130. The external communication device may be a smartphone, a desktop computer, a tablet, a Personal Digital Assistant (PDA), a smartwatch, or the like.

The light-transmitting portion 110 and the detection probe 170 are located at opposite ends of the housing 102 along its longitudinal axis. The flexible circuit board 122 and light-transmitting portion 110 are also mounted on opposite sides of the second support plate 182. Light-transmitting holes formed in the second support plate 182 align with the respective light-emitting elements 132, ensuring close attachment of the flexible circuit board 122 and enhancing space efficiency and integration.

In an embodiment, the physiotherapy device host 100 is equipped with an intelligent control architecture that enhances therapeutic effectiveness by integrating real-time sensing, dynamic adjustment, and user feedback functionalities.

The detection probe 170 in the physiotherapy device host 100 is configured to monitor various skin parameters of a user. These parameters may include, but are not limited to, hydration levels, oil content, temperature, and impedance. The detection probe 170 may comprise a combination of moisture sensors, temperature sensors, impedance sensors, or optical sensors. The detection probe 170 is located at an exposed region of the housing to maintain consistent contact with the user's skin during operation.

In one embodiment, the detection sensor is configured to analyze the hydration levels of the skin in real-time. The measured data is transmitted to the control unit for processing.

The control unit is operatively connected to both the detection probe 170 and the one or more stimulation elements 130. The control unit is configured to regulate the operation of the stimulation elements (e.g., light therapy, vibration, micro-current, or ultrasonic stimulation) based on predefined treatment parameters, such as user-selected intensity levels or durations, and dynamically adjust output levels of the stimulation elements in response to the real-time skin parameters detected by the sensor. For instance, if the skin hydration is below a target threshold, the control unit may increase micro-current intensity or phototherapy duration.

The control unit may be preprogrammed with multiple treatment modes, including preset therapy cycles tailored for different skin types or conditions (e.g., oily, dry, sensitive, or combination skin). These treatment profiles allow for rapid user selection without manual calibration.

In further embodiments, the control unit is capable of storing treatment history and user-specific data in internal or cloud-connected memory. This data is used to generate customized therapy recommendations for future use, thereby personalizing the therapeutic experience.

The user interface in the physiotherapy device host comprises one or more input components such as physical buttons or a touchscreen display. The interface allows users to: select from available treatment modes; adjust therapy intensity levels, and activate or deactivate individual stimulation elements.

In a preferred embodiment, the user interface includes a touchscreen display that provides real-time monitoring of both therapy parameters (such as duration, mode, and intensity) and skin parameter readings (such as hydration or temperature levels). The display also provides visual feedback on ongoing therapy sessions and progress.

The control unit may also incorporate a wireless communication module (e.g., Bluetooth or Wi-Fi), enabling the physiotherapy device to interface with a mobile application. The app can be used to: remotely control the device; modify treatment settings; view stored data and skin analysis reports; and receive notifications and usage reminders. The mobile connectivity enhances convenience and expands the device's usability for users who prefer remote management and data access.

In an embodiment of the present invention, the skin parameters detected by the sensors in the physiotherapy device host 100 can be transferred to a mobile device using communication channel, such as NFC, IR, BLE or Wi-Fi. When the physiotherapy device 100 is brought close to the mobile device, the communication channel transfer the data detected by the beauty instrument to the mobile phone and display it on the mobile phone. The mobile phone is provide with a mobile application corresponding to the physiotherapy device host 100, and the mobile application is used to display the skin conditions detected by the sensors.

To prevent overuse and ensure safety, the physiotherapy device host 100 is configured with an automatic shut-off function. This feature automatically terminates the operation of the stimulation elements after a predefined therapy duration, based on system defaults or user settings.

This intelligent therapy system, integrating real-time skin sensing, adaptive control logic, user feedback, and mobile connectivity, provides a comprehensive and customized therapeutic experience, improving both usability and treatment outcomes for users across diverse skin profiles.

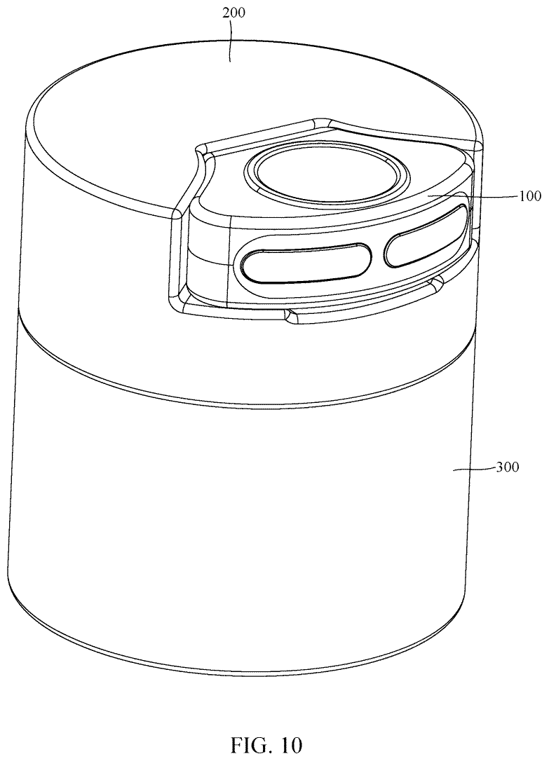

As shown in FIG. 10, the device may be integrated with a cosmetic container comprising a container body 300 and the above-described skin phototherapy device. The container body 300 includes an opening configured to receive the phototherapy device in a detachable manner, thereby sealing the container. This eliminates the need for a separate container lid, resulting in a more compact overall form factor for the integrated cosmetic container and phototherapy device assembly.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A system for providing one or more therapy to a user, the system comprising:
a physiotherapy device host, comprising:
a housing;
one or more stimulation elements disposed within the housing configured to provide one or more therapy to the user;
a circuit assembly electrically connected to the one or more stimulation elements for regulating operations;
a storage shell having a first end for attachment to a container body and a second end having a storage slot configured to receive the physiotherapy device host in a detachable manner; and
wherein the physiotherapy device host is configured to modularly fit in the storage shell.

2. The system of claim 1, wherein the one or more stimulation elements are selected from the group consisting of a phototherapy component, micro-current component, heating element, cooling element, Peltier element, vibrational element for massage, and ultrasonic wave component.

3. The system of claim 1, wherein the storage slot includes a securing mechanism selected from the group consisting of a snap-fit mechanism, magnetic attachment, friction fit, or threaded engagement.

4. The system of claim 1, wherein the circuit assembly includes a wireless communication module for connectivity with external devices.

5. The system of claim 1, wherein the storage shell is magnetically attracted or snap-fitted to the physiotherapy device host for secure attachment.

6. The system of claim 1, wherein the storage shell includes a first notch and a second notch disposed opposite to each other to accommodate components of the physiotherapy device host.

7. The system of claim 1, wherein the storage shell includes a split upper shell and a transfer piece, the upper shell is provided with the storage slot, and the transfer piece includes a mounting position to secure the device.

8. A system for providing one or more therapy to a user, the system comprising:
a physiotherapy device host having a front shell section and a rear shell section, the front shell section is wider than the rear shell section, the front shell section is provided with a light-transmitting portion, the physiotherapy device comprising:
a housing;
one or more stimulation elements disposed within the housing to provide one or more therapy to a user;
at least one detection probe to detect one or more skin parameters;
a circuit assembly electrically connected to the one or more stimulation elements for controlling therapy output; and
a storage shell having a first end for attachment to a container body and a second end having a storage slot configured to detachably receive the physiotherapy device host.

9. The system of claim 8, wherein the at least one detection probe is selected from the group consisting of a temperature sensor, moisture sensor, impedance sensor, optical sensor, or a combination thereof.

10. The system of claim 8, wherein the detection probe is configured to detect at least one of the water level, oil level, temperature, or hydration level of the skin.

11. The system of claim 10, wherein the physiotherapy device host is connected to a mobile application on a mobile device through a communication channel, and the data from the detection probe is displayed on the mobile application.

12. The system of claim 8, wherein the storage shell further comprises a secondary compartment for storing additional accessories, such as replacement therapy heads or power cables.

13. The system of claim 8, wherein the light-transmitting portion houses metal electrodes that are electrically connected to the main control board for delivering micro-current therapy.

14. A system for providing one or more therapy to a user, the system comprising:
a physiotherapy device host, comprising:
a detection sensor configured to detect one or more skin parameters of a user;
one or more stimulation elements configured to provide one or more therapy to the user;
a control unit configured to:
regulate operation of the one or more stimulation elements based on predefined treatment parameters;
adjust output of the one or more stimulation elements dynamically in response to real-time skin parameters; and
a user interface having at least one input control to allow a user to select treatment modes, adjust intensity levels, and activate or deactivate the one or more stimulation elements.

15. The system of claim 14, wherein the detection sensor is configured to analyze skin hydration levels and adjust therapy parameters accordingly.

16. The system of claim 14, wherein the control unit is programmed with multiple treatment modes, including pre-set therapy cycles based on skin conditions.

17. The system of claim 14, wherein the control unit is configured to store user treatment history and provide customized therapy recommendations.

18. The system of claim 14, wherein the system further comprises a mobile application that allows a user to control the physiotherapy device remotely via Bluetooth or Wi-Fi connectivity.

19. The system of claim 14, wherein the user interface includes a touchscreen display configured to provide visual feedback on treatment settings and progress.

20. The system of claim 14, wherein the physiotherapy device host features an automatic shut-off function after a predefined therapy duration to prevent overuse.
